SEQUENCE LISTING (3 sequences)

SEQ ID NO: 1 - DNA, 18 bp, Artificial Sequence
  Description of Artificial Sequence Synthetic polynucleotide sequence
ttagggttagggttaggg

SEQ ID NO: 2 - DNA, 24 bp, Artificial Sequence
  Description of Artificial Sequence Synthetic polynucleotide sequence
ttagggttagggttagggttaggg

SEQ ID NO: 3 - DNA, 30 bp, Artificial Sequence
  Description of Artificial Sequence Synthetic polynucleotide sequence
ttagggttagggttagggttagggttaggg

(19) United States
(12) Patent Application Publication
(10) Pub. No.: US 2004/0265996 A1
(43) Pub. Date: Dec. 30, 2004

(54) Method for the preparation of isolated cell cultures, culture meidum for the cultivation of cell cultures, and cell cultures

(76) Inventors: Johannes Schwarz, Leipzig (DE); Sigrid Schwarz, Leipzig (DE)

Correspondence Address: Reed Smith, Suite 1100-East Tower, 1301 K Street N W, Washington, DC, 20005-3373, US

(21) Appl. No.: 10/484,305

(22) PCT Filed: Jul. 19, 2002
(86) PCT No.: PCT/EP02/08086

(30) Foreign Application Priority Data
Jul. 20, 2001 (DE) 101 34 667.0

(51) Int. Cl. C12N 5/08

(52) U.S. Cl. 435/368

(57) ABSTRACT

The invention describes a novel method for cultivating cell cultures comprising a plurality of progenitor cells, wherein the method comprises one or several steps selected from the group of expansion of the progenitor cells and modification of the progenitor cells of the cell culture in a culture medium. To ensure a rapid and continuous cultivation of these particular cells, it is suggested to use a cell culture provided essentially in the form of single cells and/or agglomerates with weak cell-cell interactions caused by the external influence of the culture medium leaving the majority of progenitor cells remains intact when the agglomerates are to be converted/transformed into single cells.

Preferably the expansion and/or modification of the progenitor cells occurs under cell culture conditions which block at least partially the cellular receptors responsible for intercellular adhesion.

The culture medium can comprise a Ca²⁺ concentration of ≦0.5 mmol/l and/or inhibitors, important for cell-cell interactions of cell specific membrane bound receptors.

The invention relates to a method for the cultivation of cell cultures, particularly for the preparation of isolated cell cultures comprising a plurality of cells, wherein the method includes one or several steps selected from the group of expansion and modification of the cell culture in a culture medium. Furthermore, the invention relates to a culture medium for the expansion or modification of a cell culture having a plurality of cells. Furthermore, the invention relates to a cell culture consisting of a plurality of cells which was obtained from a culture medium where a plurality of cells were expanded and/or modified.

Conventionally, cell cultures are expanded in a culture medium, i.e the cell number in the cell culture is increased and/or modified through cell multiplication, whereby one or more cells are placed into the expansion medium, which conventionally includes the components which stimulate or are essential for cell multiplication or modification. In cell cultivation, the formation of intercellular cell-cell contacts occurs in the culture medium once a certain cell concentration has been reached, whereby a plurality of cells combine to form agglomerates, which often can only be separated with the help of digestive enzymes, resulting in a significant cell loss.

Particularly, the agglomerates formed by postmitotic neuronal cells, or their precursor cells (so-called progenitor cells), respectively, are called neurospheres. These neurospheres are currently the most wide-spread expansion form of neuronal progenitor cells. Such neurospheres grow in suspension cultures, whereby the invention relates in particular to neurospheres where the cell growth particularly depends on EGF (epidermal growth factor) and FGF (fibroblast growth factor) and/or LIF (leukemia inhibitory factor); CEE (chicken embryo extract) however is not needed for cell growth. The occurrence of such agglomerates or “spheres” is not limited to neurospheres; they can also be observed with other cell types.

The expansion of progenitor cells, especially of neural progenitor cells up to this point took place via the expansion of such agglomerates. These aggregates form a relatively compact tissue involving from a few to up to several millions of adhering cells (the diameter of an agglomerate is typically 0.01-5 mm), where particularly the core of these agglomerates (or cellular spheroids) is prone to differentiation or necrosis. As a rule, these central areas of the spheroids are known to be lost in the early stages of digestion of these agglomerates. A reduction of the spheroids leading to suspensions of single cells is of utmost necessity for many culture techniques (such as subcloning, transfection, cell sorting, cell counting). Furthermore, due to the formation of these aggregates, membrane receptors which are located within the aggregate and thus are not in direct contact with the culture medium are not very accessible. On the one hand, this impedes the multiplication of the cells due to the obstructed access of the nutritional medium, and in addition, the cells are also less accessible to manipulation by exogenous factors, which for example should cause the differentiation of the cells or other transformations of the cells.

An expansion and/or modification of cells that is as efficient as possible, is an important condition for many areas of application, for example in the preparation of neural progenitors as a resource for the restaurative therapy of neurological diseases like Morbus Parkinson, Morbus Alzheimer or others, as described for example in WO 00/78931. There is therefore a general need to increase the effects of culture and manipulation media on the cells in the culture medium, and render them more consistent.

Thus, the technical problem underlying the present invention was to provide a method for the expansion and/or modification of progenitor cells occurs and more consistent with respect to the total number of cells present in the culture medium. Another problem underlying the invention to prepare a cell culture which facilitate a faster proliferation particularly of progenitor cells, which tend to form agglomerates, and which also makes the cell modification by application of exogenous factors more straightforward.

The solution of said technical problem is achieved by providing a method for culturing progenitor cells, especially for the preparation of isolated cell cultures of progenitor cells, where the progenitor cells are present during the expansion and/or modification to a significant extent as single cells and/or agglomerates with weak cell-cell interactions, particularly with weak cell-cell interactions between progenitor cells, with the result that the formation of aggregates that comprise progenitor cells with clear cell-cell contacts is avoided. On the other hand, cells which up to now have been expanded as spheroids, especially progenitor cells and/or neuronal cells, can be transferred to single cell suspensions in the course of the transfer to the nutritional media of the invention, whereby the adhesive cell-cell interactions of the agglomerates can be disrupted by an external influence upon the culture medium, while the majority of progenitor cells remain intact during transfer of the agglomerates into single cells.

The expansion and/or modification of the progenitor cells and/or neural cells can be performed more easily and more consistently on cells which are present as single cells and/or as agglomerates with the above described weak adhesive cell-cell interactions, because the nutrients and other active compounds such as growth factors, coenzymes, plasmids, vectors and the like are more readily available to the cells. This can have the effect of an accelerated proliferation of the cells compared to cells present within spheroids. The same is valid for cells present in agglomerates with weak cell-cell interactions.

The cell culture of the present invention or the cell culture used in the method of the present invention particularly contains partially, preferably more than 25% or more than 50%, particularly preferred more than 75% or more than 90% (in every case applied to the total number of cells of the culture or of a representative sample) or essentially exclusively, i.e. more than 98% of progenitor cells and/or not immediate and/or immediate descendants of these progenitor cells, which are further or finally differentiated.

Progenitor cells according to the present invention are pluripotent cells different from omnipotent stem cells, capable of cell division, which can differentiate into certain or only certain cell types under the influence of exogenous factors. In the presence of varying exogenous factors or the conditions of their application, cells that have undergone a qualitatively or quantitatively varying differentiation can give rise to a limited number of possible cell types. The resulting cells, having been exposed to different exogenous factors, can differ quantitatively or qualitatively in their composition, for example in a way that after the effect of a first exogenous factor a first cell type predominantly develops, and after the effect of a different exogenous predominantely only a different cell type.

In particular, the neural progenitor cells in the meaning of the present invention encompass cells which can differentiate exclusively, or under certain culture conditions predominantly into neuronal cells and/or glial cells, including astrocytes or oligodendrocytes. The neuronal cells may exclusively or preferably comprise one or more neuronal cell types selected from the group of dopaminergic, cholinergic, serotonergic and/or GABAergic neurons, whereby the proportions of the cell types may vary as a function of exogenous factors or the conditions of their application. The growth of the cells according to the present invention may be dependent on EGF (Epidermal Growth factor) and/or FGF (fibroblast growth factor) and/or LIF (leukaemia inhibitory factor), but the cells do not require CEE (chicken embryo extract) in order to grow.

The progenitor cells differentiate in the presence of the commonly used nutrient solutions and with sufficient time, after transplantation into host animals, including humans, or after sufficiently long contact with containers made from glass, plastics and the like, materials commonly used for culture dishes.

Furthermore the term neural progenitor cells includes all progenitor cells that are obtainable from the brain, whereby a specific application excludes stem cells, especially embryonal stem cells. The invention however also comprises progenitor cells of other types, such as cells that give rise to muscle cells, hepatic cells or skin cells.

Neural cells within the scope of the present invention are preferably post-mitotic cells. Neural cells and neural progenitor cells may, within the framework of the invention, also be called neuronal cells and neuronal progenitor cells, respectively.

Preferably, the cell cultures of the present invention are made available, as well as the cell cultures used in the methods according to the invention, respectively, are of the type where the method steps of the expansion and/or modification do not concern tumour cells. Preferably, the cell cultures of the present invention and the cultures used in the method according to the present invention are virtually free of tumour cells, i.e. they contain less than 5% of tumour cells with respect to the total number of cells or no detectable parts. The term tumour cell includes benign as well as malignant (metastasizing tumour cells with inflitrating growth) tumour cells.

The term cells within the scope of the present invention refers to progenitor cells, especially neural progenitor cells, unless something different is either explicitly stated or arises from the context.

The term cell-cell contact within the scope of the invention refers to contacts between progenitor cells or contacts between progenitor cells and other cells, especially further differentiated or completely differentiated cells, especially further or completely differentiated neural cells, or contacts between neural cells, unless something different is either explicitly stated differently or arises from the context.

The term cell modification within the invention covers any modification of a characteristic of a cell, especially with respect to a subsequent expansion and/or differentiation, including an alteration with respect to the expression of a gene. A modification can particularly take place via a differentiation, particularly a partial differentiation, a priming, a genetic manipulation, such as transfection or similarly generally known methods.

The term cell-cell contact within the scope of the invention covers direct cell-cell contacts, where the cells adhere to each other due to direct cell-cell interactions, for example via adhesion proteins such as cadherin, selectin and/or immunoglobulins, without being limited thereto. The method according to the invention results in the reversal/prevention of cell-cell contacts with homotypical interactions or with heterotypical interactions, in order to avoid the formation of spheroids, whereby those cell-cell contacts are initial/early contacts which precede a cellular connection that stabilizes the tissue, allows a exchange of components, or has other characteristics, which allow cells to complement the actions and effects they have on each other, such as the formation of tight junctions, demosomes or gap junctions.

Furthermore, cell-cell contacts within the scope of the invention also include indirect cell-cell contacts, where the cells are connected to each other at least partially through an extracellular matrix. An extracellular matrix according to the invention is represented by an accumulation of released proteins and carbohydrates, which fills the space between the cells of an animal tissue and which may comprise collagens and/or progeoglycans. Generally speaking, each organic or inorganic material with increased structural stability compared to the culture medium, material divided from the culture medium via phase separation may be considered as a matrix, such as organic tissue materials, particularly animal cell tissue, inorganic structural materials or structural materials such as glassware walls of any kind.

The cellular agglomerates present, used or comprised in the method and cell cultures of the invention, are preferably composed of less than 100 cells, in particular progenitor cells, particularly preferred 2 to 16 cells, especially progenitor cells, per agglomerate. The agglomerates may be separated into single cells through weak external influences, especially through weak mechanical influences. The separation of agglomerates through weak mechanical influences may be accomplished through simple pipetting, through agitating at low speed, for example in the range of 50 to 250 revolutions per minute, whereas higher and lower speed can be used if necessary, through ultrasound or other suitable means, provided that the majority of the cells in the cell culture, especially of the progenitor cells, remains undamaged. The external influence on the agglomerates intended to separate them occurs preferably in a way that a damaging of the cells, especially of the progenitor cells, occurs only to a limited extent (preferably in <20 or <5 to 10% or <1 of the cells, and progenitor cells, respectively), particularly preferred is the absence of significant cell damage. Cell damage is assumed if the cells are noticeably influenced in their proliferation or differentiation in response to the external influence, or if the cell membranes are destroyed.

The cultivation, i.e. the expansion and/or modification of the cell culture preferably occurs with a cell culture where the proportion of the cells which is present as single cells or cellular agglomerates with weak cell-cell interactions with respect to the total number of cells in the culture consists of more than 25%, preferably more than 50%, especially preferred more than 75%, particularly more than 95% or more than 99%, or where virtually all the cells of the cell culture are present as single cells and/or agglomerates with weak cell-cell interactions.

Particularly preferred is the case where the proportion of cells present as single cells is more than 25%, preferably more than 50%, more than 75% and particularly more than 95% or more than 98% up to virtually 100%. The above statement concerning the proportions of the cells with respect to the total number of cells refers to the proportion of progenitor cells, as the proportion of neural progenitor cells or alternatively as the proportion of neuronal cells.

The cells present as single cells and/or in form of agglomerates with weak cell-cell interactions, particularly progenitor cells, especially neural progenitor cells, or neuronal cells, may be present in the culture medium in amounts from 100 to 10,000,000 cells or more/ml culture medium, preferably 1000 to 1,000,000 cells/ml culture medium, especially preferred 10,000 to 500,000 cells/ml culture medium. Particularly, up to from 100,000 to 500,000 cells/ml culture medium may be present.

The cultivation of the progenitor cells and/or neural cells is preferably done under conditions which at block the cellular receptors responsible for intercellular adhesion at least partially. For example, a block may be accomplished by preventing the expression or activation of the receptors responsible for cell-cell adhesion under culture conditions by preventing the access of a substance which is necessary for activation of the receptors, by not adding it to the culture medium, or through the addition of inhibitors to the culture medium, which prevent the binding of the activators to the receptors. Additionally or alternatively substances can be added to the culture medium which result in an immediate receptor block, for example if such compounds bind to the receptors and hereby prevent cell-cell adhesion. Furthermore, the term receptor blockade also includes means leading to the degradation, especially the selective degradation, of the receptors.

The method steps according to the method of the invention, particularly of the expansion and/or modification of the cells or progenitor cells, respectively, especially neural progenitor cells, occurs preferentially on cells present in a cellular stage allowing the expression of adhesion molecules under suitable culture conditions, especially e-NCAM, (psa/NCAM) and/or N/Cadherin and/or L1. Accordingly, the cell cultures according to the invention relate preferably to cultures where the cells are present in a cellular stage allowing expression of adhesion molecules under suitable culture conditions, especially e-NCAM, (psa/NCAM) and/or N/Cadherin and/or L1.

Preferably, the manipulation of the progenitor cells and/or neural cells occurs under culture conditions where more than 25%, preferably more than 75% and particularly preferred more than 95% of the receptors specific for the cell-cell adhesion and/or for the multi adhesion proteins are blocked. Particularly more than 99% or practically all receptors may be blocked.

The manipulation of the cell culture may particularly occur under conditions where the cellular receptors of the progenitor cells and/or neural cells which are specific for adhesion molecules and which generate direct cell-cell contacts, are partially or completely blocked. Such cellular receptors, which may be integrated within the cell membranes of the respective cells, may particularly be cadherins, selectins, integrins and/or receptors of the immunoglobulin (Ig) superfamily, particularly NCAM, also particularly embryonal NCAM (e-NCAM or psa-NCAM) and/or ICAM and/or L1, without being limited to these exampes.

For example, psa-NCAM can be effectively inactivated by endoneuraminidase, respectively its expression can be reduced through inhibition multi-adhesion of NF-kappa-B. The culture medium preferably comprises effective amounts of enzymes capable of digesting carbohydrates such as endoneuraminidase, in order to block psa-NCAM more than 10 or more than 25%, preferably more than 75%, and especially preferred more than 99% or more than 98% up to virtually a complete block. The other cellular receptors that were mentioned above, especially N/Cadherin and/or L1, may be blocked accordingly by suitable inhibitors.

Furthermore, the manipulation of the cell cultures, alternatively or cumulatively, may occur under conditions where the cellular receptors which are specific for proteins are partially or completely blocked. Such multi adhesion proteins which are present in the extracellular matrix and are able to interact with collagens and proteoglycans, are for example fibronectins, which are capable of adhering to cell surfaces by means of specific integrins.

An advantageous embodiment of the method according to the invention for the production of isolated cell cultures consists of the expansion and/or manipulation of the progenitor cells and/or neural cells of the cell culture occuring in a culture medium, which has an effective Ca2+ concentration of ≦1.0 mmol/l, preferably ≦0.5 mmol/l culture medium, more preferably ≦0.1 mmol/l culture medium. Preferably, the total concentration of Ca²⁺ ions in the culture medium is identical the effective concentration. If needed, a masking of Ca ions through suitable masking agents which decrease the concentration of free Ca ions capable of binding to the receptors responsible for cell-cell adhesion, may occur.

For this purpose, chelating agents like EGTA, EDTA, Kronenether and others may be used.

If needed, the culture medium may be free of Ca²⁺ ions apart from unavoidable contaminations; preferably, the medium is not Ca²⁺ free. A minimum amount of Ca²⁺ ions of 0.001 to 0.1 mmol/l, particularly 0.01 or 0.05 to 0.1 mmol/l culture medium, has been proven to be advantageous.

Furthermore, the culture medium comprises preferably Mg ions in only low concentration, or it is free of Mg ions, apart from unavoidable contaminations. The Mg concentration of the culture medium can be ≦2 mmol/l culture medium, preferably ≦0.6 mmol/l or ≦0.1 mmol/l culture medium.

In order to block the receptors which are specific for the formation of adhesive cell-cell contacts, the expansion and/or modification of cells can occur in presence of inhibitors (such as receptor antagonists, antibodies to receptors or antisense to corresponding receptor RNA).

Particularly, the cultivation of the cell medium may occur with a culture medium which contains effective amounts of one or more inhibitors, which are specific for cadherins, selectins, integrins and/or immunoglobulins (Ig family), especially for eNCAM, N-Cadherin and/or L1. These inhibitors bind directly to the receptors and thus block the cell-cell adhesion. Specific inhibitors for E-, P-, N-cadherin are preferred, but the culture medium may also comprise specific inhibitors for other types of cadherins. The culture medium may comprise alternatively or additionally inhibitors of NCAM receptors (especially eNCAM) and/or of the ICAM family and/or for L1. The inhibitors may be present in the culture medium in a concentration that is sufficient to block all or a desired proportion of the receptors.

The inhibitors can be present either individually and/or in the presence of several inhibitors as a total amount, in concentrations from about 0.001 to about 10 micromole/l culture medium, for example 0.01 to 1 micromole/l or 0.2 to 1 micromole/l. The inhibitors can also be present, if needed, in higher or lower concentrations, for example depending on the Ca²⁺ concentration of the culture medium, as long as a sufficient block of the receptors is produced.

The culture medium may also have a low concentration of Ca²⁺ ions, for example ≦0.1 mmol/l culture medium in the presence of inhibitors that are specific for cell-cell adhesion receptors, in order to adjust the proportion of single cells and/or agglomerates with weak adhesive cell-cell interactions with respect to the total number of cells in the culture medium.

It is especially preferred when the manipulation of the single cells and/or cellular agglomerates with weak cell-cell interactions occurs in the presence of a high telomerase activity of the cells. The method according to the present invention allows for an at least two fold increase of telomerase activity compared to progenitor cells obtained from rodents or human tissue, under otherwise identical conditions, and this method additionally prevents the reduction of the telomerase activity in neural progenitors from human tissue, which was observed in the previous culture techniques.

The use of culture media which are low on calcium is especially advantageous here, since the low calcium content, besides the fact that there occurs no activation of adhesion molecules, has the effect of causing very little or no telomerase inhibition. The telomerase inhibition can consist of less than 50%, 75%, or 90% with respect to the fully activated telomerase present in the culture medium, preferably less than 25%, more preferably less than 10%. Telomerase is a ribonucleoprotein which catalyses telomere repeats of all 3′ ends of cDNA, which are getting lost during cell division. The high telomerase activity shortens the cell cycle and at the same time prevents or diminishes the senescence of the cells.

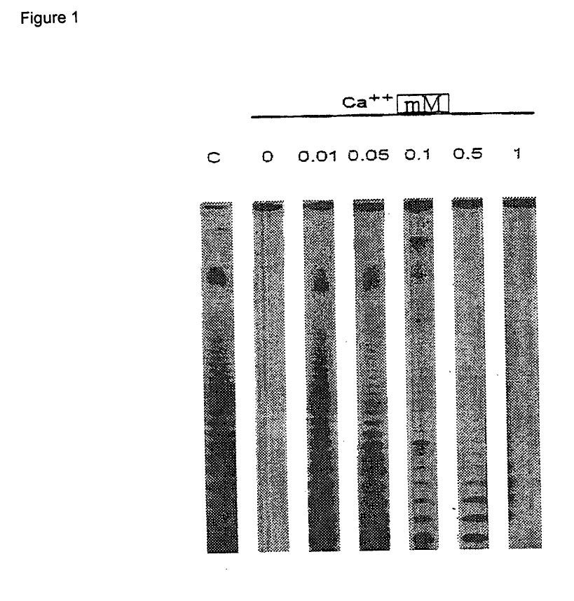

For example, the telomerase activity can be determined, with the use of a PCR ELISA, following the “Telomeric Repeat Amplification Protocol (TRAP). As indicated in FIG. 1, a TRAP assay of the cell culture of the present invention having a Ca concentration of 0.01 to 0.5 mmol/l, especially in the area of 0.01 to 0.1 mmol/l, shows a distinct telomerase activity compared to a control sample of a tumour cell line. In the presence of a high Ca2+ concentration or in a Ca2+ free medium, only a weak or no telomerase activity is observed.

The telomerase activity is measured preferably by using an enzymatic luminometric PPI assay (ELIPA) (Xu S Q et al, 2002, Bioluminescent Method for Detecting Telomerase Activity, Clinical Chemistry 48:7, pp. 1016-1020). The prolongation of the telomerase repeats according to the “Telomeric Repeat Amplification Protocol” (TRAP) is catalysed by telomerase, which frees 6 PPI for each TTAGGG repeat. The liberated PP1 quantitatively forms in combination with adenosin-5′-phosphosulfate adenosine-tri-phosphate (ATP) when ATP-sulfurylase is added. The luciferace luminescence system is based on the measurement of the amount of light formed by the luciferase-catalysed reaction of ATP and luciferin. The light emission is time-independent and proportional to the amount of ATP formed.

Using a telomerase ELIPA assay with a cell culture according to the present invention having a Ca²⁺ concentration of 0.01 to 0.5 mmol/l, especially in the area of 0.01 to 0.1 mmol/l, a distinct telomerase activity is observed compared to a control sample C of the tumour cell line A549 (CA cells of the lung, Xu et al, 2002).

In tumour cells a distinct telomerase activity is observed. In the presence of very high Ca²⁺ concentrations or in Ca²⁺ free medium however, only a weak or no telomerase activity is observed.

Through adjusting the Ca²⁺ ion concentration or through other suitable means, the method of the invention allows to obtain progenitor cells of mammals, especially human progenitor cells, which have a telomerase activity of above 20%, preferably above 33%, more preferably above than 50%, more preferably above 75%, and most preferably above 90% of the telomerase activity compared to the control sample of the tumour cell line. These cells may be in particular neural progenitor cells.

The method of the invention and the culture medium according to the invention may particularly be used, without being limited thereto, in combination with the method disclosed in WO 00/78931, which is based on the approach to keep neuronal progenitor cells in culture and to multiply them. After sufficient expansion, these cells can be differentiated secondarily into specific neurons, such as dopaminergic neurons, by using suitable active agents.

Independently thereof, cell cultures may be used or obtained in a method step according to the method of the present invention, especially in the case of a partial differentiation, wherein the differentiation conditions disclosed in WO 00/78931 are included herein by reference, and/or in the case of a modification and/or expansion and/or selection of cells, or cultures or cell culture media according to the invention can be obtained, which consist of more than 25% or more than 50%, preferably more than 75% or practically exclusively of the desired neural cells (one or more neural cell types), or their immediate precursors, and where other cells, especially immunocompetent glial cells, are only present in proportions of <90%, <95%, <99%, preferably <10%, more preferably <5% or <2% with respect to the total number of cells in the cell culture.

Especially preferred are glial cells only in amounts which do not show a physiological effect anymore, and which in particulary are not detectable. Immediate precursors within the scope of the invention include cells which undergo final differentiation immediately after transplantation into a host tissue, contact with a surface of a commonly used tissue culture dish such as a glass wall and/or the transfer into a nutritional medium of commonly used for the cultivation or multiplication of cells. The method described herein allows for a further improvement of the method of WO 00/78931.

Particularly, the method for the preparation of a growth capable cell culture from precursor cells, comprising the following method steps, particularly in the following order:

obtaining brain parts from a mammal

selection of precursor cells

expansion of the precursor cells, if necessary immediately following a selection

partial differentiation of the progenitor cells, if necessary selection and if desired subsequent expansion of the selected cells,

if necessary one or more repetitions of one or more of the steps of expansion, selection and/or partial differentiation, allows for the fact that the precursor cells of the cell culture may be present at least during one, preferably during all of the above mentioned method steps of the expansion and/or partial differentiation and/or selection, to a significant proportion (i.e. to more than 25%, more than 75%, more than 90%, more than 95% or more than 98%) as single cells and/or agglomerates with weak cell-cell interactions, whereby the cell-cell interactions are interruptable through the application of external influences on the culture medium whereby the majority of the progenitor cells remain undamaged during the transformation of the agglomerates into single cells. In particular, the cells of the invention are present immediately after the sequential steps of the expansion and partial differentiation, if needed also during the immediately subsequent selection, as the above defined single cells or weak cell-cell agglomerates. A final differentiation may follow the above listed steps.

The method of the invention allows to obtain cell material in a more simple and reproductive manner; respectively a cell culture according the invention may be made available which consists of more than 25% or more than 50%, or more than 75% or virtually exclusively dopaminergic neurons and/or cholinergic neurons and/or GABAergic striatal and/or serotonergic neurons, separately or in combination; i.e. the proportion of the mentioned neurons with respect to the cell material is higher than 90%, preferably higher than 95%, or higher than 99%, respectively does not contain physiologically active amounts of other cells, especially glial cells.

The neural progenitor cells used according to the invention, which through their multiplication, selection, and initially partial and subsequently final differentiation allow for the preparation of cell cultures and cell material which can be transplanted, can be obtained from fetal as well as adult neural cell material (brain, preferably midbrain or bone marrow) of a mammal including a human. The obtained brain parts may particularly originate from brain parts which contain the type of neurons into which the progenitor cells will differentiate partially or completely, or to brain parts where the progenitor cells are applied to for the treatment of a brain dysfunction. The adult cell material is advantageously prepared from periventricular sections. The fetal material may be prepared from fetuses aged 3 to 25 weeks, preferably 5 to 11 weeks, or 6 to 20 weeks after fertilization. In particular, fetal tissue may be obtained after the completion of fetal development up to the 12th week of pregnancy. As a rule it is not tissue from human embryos. Alternatively, also mesenchymal tissue was used. The neuronal progenitor cells may also be obtained from stem cells of the blood from umbilical cord tissue. Concerning the isolation and cultivation of cells, reference is made to Daad, Weiss, J. Neursci. 1999; Magrassi et al. Developement 1998, 54 :105-115; Ptak et al. Cell Transplant 1995, 4:299-310; Liepelt et a. Brain Res. Dev. Brain Res. 1990, 51:267-278, Buc-Caron, Neurobiol Dis 1995, 2 :37-47, Svendsen C N et al., Exp. Neurol 1997, 148 :135-146; Sah et al., Nat Biotechnol 1997, 15 :574-580; Chalmers-Redman et al. Neuroscience 1997; 76 :1121-1128, all incorporated herein by reference in their entirety.

According to the invention, transplantation capable neural cell material may be obtained through a method which comprises an expansion of the human progenitor cells which was immediately or mittelbar obtained from mammalian cell material, including humans, a partial in-vitro differentiation, and a selection, whereby the finally obtained neural cultures are undergo differentiation into the final cell type to a high percentage without addition of other factors or genetic manipulation, or differentiate after transplantation. If needed, a repeated expansion of the cellular material may follow after a partial differentiation and selection step of the progenitor cells, the steps of the partial differentiation of selection can be repeated several times, whereby the type of differentiation can vary.

The invention makes it possible to select to an extent and differentiate neural progenitor cells, in a way that predominantely one specific cell type develops after the addition of nutritional medium, contact with a wall of a generally used culture dish, or after transplantation, respectively.

The method of the invention, for example as improvement/further development of the method disclosed in WO 00/78931, without being limited thereto, can comprise one or several steps of the cell modification in the form of a partial or complete differentiation and cell selection.

One, several or all method steps of the differentiation, especially of the partial differentiation and/or selection can be executed in a medium which contains partially or virtually exclusively progenitor cells, especially neural progenitor cells, in form of single cells and/or agglomerates with weak cell-cell interactions. Alternatively or additionally, one, several or all method steps may be untertaken in a medium which is free of isolated precursor cells and/or agglomerates with weak cell-cell interaction, or contains only negligible amounts with respect of the total number of cells in the medium.

The partial or final differentiation which may occur under in-vitro conditions, may occur in progenitor cells, especially in neuronal progenitor cells, whereby progenitor cells of other cell types may also be used, for example cells that give rise to muscle cells, liver cells or skin cells, without being limited thereto. The differentiation, especially the partial differentiation, does not only occur significantly faster under these culture conditions, but also more reproducibly and more selective compared to a final differentiation or partial differentiation in the presence of spheres.

It is especially advantageous to use the culture media of the present invention containing single cells and/or agglomerates with weak cell-cell interactions during the partial differentiation of progenitor cells through priming and/or genetic manipulation, especially transfection (for example transient and nontransient transfection), whereby the partial and final differentiation may also occur in turn under hypoxic conditions, such as described below.

A partial differentiation of the cells may occur especially through treatment with one or more compounds selected from the group of cytokines, growth factors, transcription factors, neurotransmitters, hormones and gangliosides, which in particular may also be used in a priming step. The partial differentiation of neuronal progenitor cells is in particular described in WO 00/79931, whose content in respect to the above mentioned components is incorporated herein by reference.

Possible growth factors to be used include one or more selected from the group of epidermal growth factor (EGF), especially EGF1, EGF2, EGF3 with the subgroups alpha and beta, transforming growth factor (TGF) alpha and beta, LIN-3-protein, fibroblast growth factor (FGF), FGF1 and FGF2, nerve growth factor (NGF), brain-derived neurotrophic factor (BNDF), neurotrophine (NT), especially NT-3, NT-4, NT-5, NT-6, insulin-like growth factors (IGF), especially IGF-1 and IGF-2, glial cell line-derived neurotrophic factor (GDNF), neurturin (NTN), persephin (PSP), vascular endothelial growth factor (VEGF), including their subgroups or factors with similar effects.

Possible cytokines to be used include one or more selected from the group of interleukins (IL 1-16), leukemia inhibitory factor (LIF), ciliary neurotrophic factor (CNTF), tumour necrosis factor (TNF), especially TNF alpha, interferons (IFN), especially IFN alpha, macrophage inhibitory or stimulating factors, especially macrophage migration inhibitory factor (MIF), mitochondrial import stimulation factor (MSF) and retinic acid.

Possible neurotransmitters to be used include one or more selected from the group of dopamine, acetylcholine, GABA, glutamate, glycine, taurine, proline, noradrenaline, serotonin and neuropeptides, especially substance P and enkephalin.

The neurotransmitter may be used alone or in the presence of growth factors and/or cytokines.

Hormones such as growth hormones, thyroid hormones (especially for the differentiation of progenitor cells into dopaminergic neurons), steroid hormones or gangliosides, including in each case their derivatives, may be used alone or in combination with the above listed agents and combinations of agents.

In order to produce dopaminergic neurons, especially GNDF, LIF and one or more of IL 1-11 may be used alone or in combination, especially the combination of IL-1, GNDF, LIF, IL-11 including the respective subgroups.

The exogenous factors may be used alone or in combination, in respective concentrations of 25,000 to 0.005 ng/ml, preferably 1 to 100 ng/ml expansion solution, without being limited hereto. For the differentiation is IL-1 particularly useful, in a concentration of 0.005 to 50 ng/ml or 0.005 to 10 ng/ml, preferably 0.01 to 25 ng/ml or 0.05 to 0.25 ng/ml. IL-11 and LIF may be used in turn in concentration of 0.01 to 100 ng/ml, preferably 0.5 to 2.5 ng/ml. GNDF can be applied/added in a concentration of 1 to 25,000 ng/ml, preferably 1 to 100 to 2,500 ng/ml (check UNITS).

The factors may also be added in combination using these concentrations. The concentrations to be used are however not limited to the above mentioned values, and may vary depending on the other factors to be used, among other factors.

The term partial differentiation in the form of a priming encompasses a method which comprises the treatment of (monoclonal) neural progenitor cells with one or more exongenous substances, especially one or more substances selected from the group of growth hormones, cytokines, neurotransmitters, whereby a partial differentiation of the progenitor cells in further differentiated cell types occurs, i.e. cell types where the cells have further charateristics of finally differentiated cells, such as the expression of certain genes or certain external characteristics. If need be, so-called conditioned media may also be used for this, i.e. culture media which are used in particular for the culture of neurons of a certain desired nerve cell population (for example dopaminergic neurons, cholinergic neurons, GABAergic neurons and/or serotonergic neurons or also glial cells). Those exogenous factors are then removed at a time point when the cell differentiation is still reversible, which allows for a further expansion of the cells. The cells are usually in contact with the exogenous factors from 1 to 12 hours, preferably 3 to 6 hours, in exceptional cases also for shorter or longer time intervals.

Such differentiation-reversed progenitor cells are called primed cells. During a renewed exposure to effective exogenous factors, which may also comprise the transfer of the cells to another medium, for example the transplantation into a tissue such as for example a brain, a much faster differentiation of progenitor cell occurs. In the course of a renewed expansion of the primed cells, primed monoclonal cell lines can be obtained, which already express genes which guarantee a higher specificity.

It is especially advantageous, to practice the method of the invention with progenitor cells, especially progenitor cells in the form of single cells or aggregates with weak cell-cell interactions, which are partially differentiated in a way that they remain capable to be primed, i.e. to a partial or complete reversal after removal of the medium having caused a partial differentiation, whereby the state of partial differentiation is independent of whether such a priming has indeed been completed or not. The neuronal progenitor cells are therefore present in a particularly early stage of differentiation.

In particular, in the context of the method of the invention, the culture medium of the invention can also be used for the partial differentiation through transfection, comprising amounts of single cells and/or agglomerates with weak cell-cell interactions. Such a transfection can also be used in the context of the above described priming, or alternatively thereto. Through the transfection (also called transformation or transduction), which comprises, independently of the specially used method, an insertion or a transfer of a gene or of genes into the cells, can favour a differentiation of the progenitor cells into a selected/desired cell type, especially into a specific neuronal type. Included therein is also the transient expression of such genes which does not alter the hereditary material of the cells and nor incorporates foreign genes after a transplantation into the tissue, for example the brain, but determines the further fate of the cells.

Possible genes that may be used include the ones specific for certain neural cell types, whereby the genes listed in WO 00/79931 in the chapter “Partielle Differenzierung durch Transfektion” (Partial differentiation through transfection) are included herein by reference. For example, the development of dopaminergic neurons can be steered through the transfection of genes which code for members of the steroid or thyroid hormone receptor family such as tyrosine hydroxylase or receptors for Nurr-1 and/or Nurr-77, or through genes coding for the vesicular monoamine transporter or for the dopamine transporter, generally genes which are specific for dopaminergic neurons In order to select cholinergic neurons, genes which are specific for these neurons may be transferred, especially genes coding for the nicotinic acetylcholine receptor, especially presynaptic alpha and beta subunits, especially alpha-7, genes coding for the nerve growth factor (NGF) receptor or for cholinesterase. The partial differentiation of striatal neurons can be steered through genes coding for the gamma-amino butyric acid (GABA) transporter, through genes coding for dopamine receptors, genes coding for glutamate receptors, enkephalin or substance P.

A transient or stable transfection is made possible through commercially available transfection reagents (for example effectene, qiagen). The corresponding DNAs are amplified out of the human genomes with the use of polymerase chain reactions. The transfection occurs with the standard methods of the literature. This allows for a transient or stable transfection of progenitor cells.

The method step of cell selection, especially the selection of progenitor cells, may preferably occur through a subcloning step or another suitable method. The subcloning may especially occur using one or more methods that are executed in a timely suitable fashion, selected from the group of: subcloning through final dilution, especially a plating out of single cells; subcloning through micromanipulation of marked vital cells; subcloning through fluorescence-activated cell sorting of marked vital cells; subcloning through magnetic concentration of magnetically marked cells, without being limited thereto. Independently thereof, reference is made, with respect to the execution of the selection, to the chapter “Selektion durch Subklonierung” (selection through subcloning) of WO 00/78931, which is hereby included by reference.

A cell sorting, encompassing a method which allows for the selection of a multitude of cells with similar characteristics from the cell culture, by way of a screening of a cell probe, for example through micromanipulation of marked vital cells, subcloning through fluorescence activated cell sorting of marked vital cells; subcloning through magnetic concentration of magnetically marked cells, can conveniently take place after an expansion or partial differentiation and before a further selection, for example through final dilution, in order to increase the efficiency of the method. The cell sorting can be carried out under dysoxic conditions. In the case of multiple cell sorting, different techniques may be used during the different steps, which carry out a sorting according to different characteristics of the cells. For example, a first step may represent a cell sorting according to a marker which determines the differentiation stage of cells, for example with respect to the expression of certain markers. In a second sorting step, for example after a partial differentiation, a scanning step can be carried out for a specific cell type, for example oriented towards dopaminergic neurons, whereby the order of the two aforementioned steps can also be interchanged. The second step can also entail a sorting according to other markers than the first step. It has been proven to be particularly effective to perform a first cell sorting with IL-1 alpha antibodies (for example Santa Cruz, 1.10 microg/ml) after an expansion of the cell culture, and to carry out a second cell sorting step using NCAM antibodies (for example DSHB, 1-10 microg/ml) after a subsequent partial differentiation, especially through priming.

The cells are being sorted by loading the antibodies with magnetized particles via biotinylation; they may then be absorbed onto magnetic columns (for example Mylteni GmbH).

The subcloning of the progenitor cells can carried out, in particular independent of the chosen ethod, in a way that only one cell remains in each culture dish especially when subcloning through final dilution), or that only one or several cells of a chosen cell type defined by the corresponding choice of a cell-typical marker to be used, remains in a culture dish, in cases where a cell type specific subcloning took place, for example through fluorescence marking, FACS, Immuno Magnetic Cell Sorting (IMS or MACS) concentration in combination with cell-type specific markers. The cells which are plated out in this manner may be expanded subsequently, whereby monoclonal cell lines are obtained. Mitogenic substances are preferably added to the media (see the growth factors cited previously) in order to achieve a multiplication of the single cell. The expansion, differentiation and characterization further occur in the same manner as described above for polyclonal progenitor cell suspensions. This is particularly valid independent of the chosen subcloning method.

By means of an example, it is mentioned that the subcloning of the progenitor cells may occur through micromanipulation after fluorescence marking of vital cells, by staining the living cells with a specific marker for the cell population of interest. As a marker for dopaminergic cells, the cells can be transiently transfixed, for example through marking with the gene coding for the enhanced green-fluorescence protein (EGFP, Clontech), which is expressed under the control of specific dopaminergic promoters (tyrosine hydroxylase and/or dopamine transporter promoter).

The green fluorescent cells can be cloned subsequently as described. For cholinergic cells, the same approach is used with the promoter for choline acetyltransferase (ChAT), for GABAergic neurons with the promoter of glutamyl-decarboxylase (GAD) or other suitable promoters. WO 00/78931 describes how other subcloning methods allow for the selection of specific cell types, especially dopaminergic, cholinergic or GABAergic cells can be selected, with the result that individual cells or several cells of a specific cell type are present.

All subcloning methods occur preferably in a cell stage where the cells are as differentiated as possible, without the TEILUGNSFAHIGKEIT of the cells is diminished; this means after a priming, genetic manipulation, change of the atmosphere or treatment with exogenous factors.

The above described steps of partial differentiation, selection (cloning) and/or expansion may be combined as needed and applied repeatedly.

Following the selection of progenitor cells, one or several method steps of multiplication of progenitor cells, the partial and/or complete differentiation of progenitor cells, or the renewed selection of progenitor cells, may follow.

For the final differentiation of progenitor cells, the cells can be mounted in vitro through plating on poly-L-lysin-coated cover dishes or plates having 48 cavities (“48-Lochplatten”) in neurobasal medium (Gibco). FCS, cytokines and/or striatal-conditioned media can be added. For example the cytokines IL-1a, IL-1b, IL-4, IL-11, LIF, GDNF or other exogenous factors such as described in the paragraph “priming” can be used. The cells are being differentiated during 7 to 10 days at 37° C. in a humidified atmosphere prior to the fixation and other investigations.

The functional integrity of the neurons, such as DA and GABA neurons, may be determined through measuring the uptake of triiodinated neurotransmitters. After the preincubation during 10 minutes in an incubation buffer comprising 100 microM pargylin, 1 mM ascorbate and 2 microM beta-alanine (and 3 microM GBR12909 and 1 mM 2,4-Diamino-n-butyric acid, DABA, for the determination of the non-specific uptake), 50 nM [3H]DA, [3H]Choline or [3H]GABA can be added for 15 min at 37° C. (check units and superscripts, greek letters fro micro etc). The uptake is stopped through a washing step of the plates with cold PBS, and the remaining radioactivity of the lysate is measured by using a liquid scintillation counter. The specific uptake is determined as the difference between the uptake in the absence (total) and the uptake occurring in the presence of GBR12909 and DABA (non-specific). Furthermore, the method of the invention for the cell culture can also include an expansion and/or modification of the cells, for example through a partial differentiation, especially in the form of a priming and/or selection of the cells or one or several other method steps under dysoxic conditions. Such dysoxic conditions can comprise a decrease or increase of the oxygen activity compared to air under standard conditions (21 Vol.-% O₂) or conditions which can be induced via a reduced or increased oxygen activity. The oxygen activity can correspond to an atmospheric oxygen concentration of ≦15 Vol.-%, preferably ≦5 Vol.-%, or ≦3 Vol.-%, especially preferred ≦1 Vol.-%. If needed, the nitrogen concentration of the gas which is in exchange with the culture medium may simultaneously be increased, compared to air under standard conditions, or additional gases such as CO₂ may be added, whereby a CO₂ concentration of 1 to 13 Vol.-%, preferably ca 5-10 Vol.-% may be present, without being bound to these values. Alternatively or in addition to the lowering of the oxygen concentration of the gas in exchange with the culture medium, other substances can be used which impair the gain of energy or simulate a diminished oxygen content, such as inhibitors of the mitochondrial respiration like Rotenon, MPP, malonate and others. These substances may be present in amounts corresponding to the diminution of the atmospheric oxygen concentration from 21 Vol.-% to ≦15 Vol.-%, preferably ≦5 Vol.-%, or ≦3 Vol.-% especially preferred ≦1 Vol.-%.

The invention further relates to a culture medium which may be used during the expansion and/or modification of a cell culture comprising a plurality of cells, whereas the cells preferably tend to form spheres in the culture medium. According to the invention, the culture medium may be adjusted in a way that the cells tending to form spheres, especially precursor cells such as neural precursor cells or neural cells, are present in the culture medium at least partially as single cells or agglomerates with weak cell-cell interactions. Preferably, the culture medium has the characteristics that were described for the medium used for carrying out the method of the invention, therefore it is referred thereto in order to avoid repetition. In particular, the culture medium can be adjusted in a way, that in the presence of 100 to 10 millions/ml culture medium precursor cells such as neural precursor cells or neural cells, the cells at least partially, preferably to an proportion of >25%, especially preferred virtually exclusively as single cells or as agglomerates with weak cell-cell interactions, which have preferably a size of <32 cells, whereby cellular receptors responsible for the formation of cell-cell adhesion are at least partially blocked.

In particular, the cell medium may comprise a Ca2+ concentration of <than 0.5 mmol/l culture medium, in particular <than 0.1 mmol/l culture medium, especially preferred <than 0.05 mmol/l culture medium, or it may be calcium-free, apart from unavoidable contamination. Alternatively or additionally, the culture medium may contain inhibitors of cell-cell adhesion, as described above.

The culture medium may further comprise compounds promoting expansion and/or being essential therefor, selected from the group consisting of amino acids, nucleic acids or precursors thereof, salts, vitamins, provitamins, enzymatic cofactors, hormones, growth factors, physiologically active sources of carbon, physiologically active sources of nitrogen, trace elements and/or the respective precursors. Depending on the desired culture conditions it is not necessary to add one or several of the above mentioned components to the culture medium, or the medium can contain further components.

The culture medium may comprise the compounds promoting expansion and/or being essential therefor in concentrations enabling the cells to survive in the culture medium for a time interval from 1 hour to 10 days or longer, for example at least 1-0.3 days or 5 days, without being limited thereto, preferably without significant impairment of their characteristics, and/or where they may perform 1 to 10 or more, preferably at least 3, division cycles.

For example, the culture medium may comprise about 0.00001 mmol/l soluble copper salts (for example CuSO₄), about 0.003 mmol/l soluble iron salts (for example FeSO₄), about 3-4 mmol/l KCl, about 100 mmol/l NaCl, about 15 mmol/l NaHCO₃, about 0.45 mmol/l KPO₄, about 0.9 mmol/l NaH₂PH₄, about 0.05 ZnSO₃, selenic acid, about 1-25, preferably 3-15 mmol/l, especially about 10 mmol/l glucose (whereby the designated glucose content can be advantageously independent of the remaining composition of the culture medium), about 30 mmol/l HEPES; about 0.03 sodium hypoxanthine, 0.001 to 0.003 mmol/l of each liponic acid, phenyl red, sodium putrescine, about 2 mmol/l sodium pyruvate, amino acids, biotin, vitamins and provitamins such as d-calciumpantothenate, choline, chloride, folic acid, niacinamide, pyrodoxine, riboflavine, thiamine, thymidine, vitamin B12, growth factors such as inositol or EGF; corticoids such as dexamethasone, hydrocortisone or cortisone, insulin, human albumin, choleratoxine, phosphorethanolamine, FGF (for example bFGF) or LIF. It is understood some of these components are not absolutely necessary for certain uses, or that different components commonly used in culture media may be used. Of special significance are however the components choleratoxin, cortisone, insulin and human albumin.

The culture medium of the invention may be cell-free for storage purposes. If the culture medium contains a cell culture which is comprised partially or completely of single cells and/or agglomerates with weak cell-cell interactions, usual procedures like centrifugation or other suitable methods allow the harvest of cell material which is isolated and essentially separated from the culture medium. The material cultivated in a medium low on calcium, i.e. having a calcium concentration of less than 0.5 mmol/l, is characterized in particular by a high telomerase activity.

A further aspect of the invention covers the use of a culture medium according to the invention for the preparation of a cell culture of the invention.

The progenitor cells of the cell culture of the invention differ further from the conventional neural progenitors in that a differentiation occurring during the expansion is at least diminished or completely inhibited. The expression of neuronal and glial markers (MAP2, NeuN, NCAM, GFAP, etc) which can be demonstrated in conventional spherides, is here largely suppressed.

Furthermore, the DNA fragmentation during beginning apoptosis which is observed in conventional spheroids in 5-10%, is practically absent in the cells of the present invention, i.e. less than 2%, preferably less than 1% to less than 0.5%, especially preferred not measurable. The DNA fragmentation may be determined using generally known methods (TUNEL staining, for example cell death detection kit, Roche Biochemicals).

The cell cultures of the invention do not characterized by the fact that they are more easily expandable and modifiable compared to the cultures consisting of spheroids, but they also may be used advantageously in therapeutic methods, where specific cell material is applied to patients. Such an application may occur through transplantation, for example also through infusion or other suitable means.

FIG. 1 shows a TRAP ELISA assay of tumour control tissue (positive control) and neural progenitor cells in medium with increasing Ca concentration. The telomerase activity of neural progenitor cells maximizes at a Ca concentration of 0.01 mM and 0.05 mM.

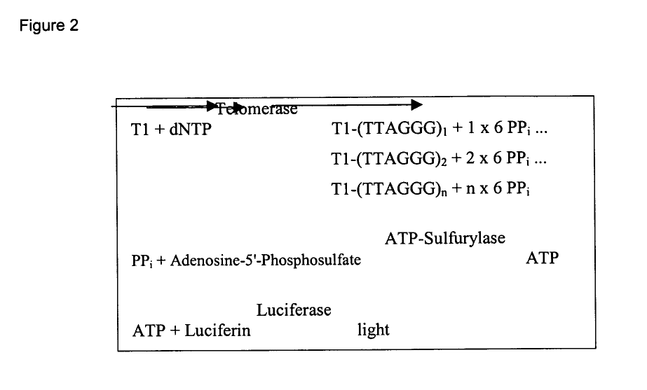

FIG. 2 shows the working principle of ELIPA..T1 Primer (5′TTAGGGTTAGGGTTAGGG-3′) for the prolongation of the telomerase repeats (after Xu et al, Clin Chem 2002).

The application of the method according to the invention and and of the culture medium is described in the following examples, which refer to such applications. For further details, it is referred to the chapter “Aligemeine Verfahrensdurchfuehrung” (General methods) as well as the example of WO 00/78931, which is hereby incorporated by reference, whereby the main differences to the example of WO 00/78931 are described as follows:

### EXAMPLE

In order to obtain neural progenitor cells, the cerebral tissue is prepared in the usual ways, followed by a homogenization of the cerebral tissue. For this purpose, the tissue is treated with a proteolytic enzyme in a suitable concentration, for example serine protease, in order to loosen the tissue connection.

A DNAse solution in a suitable concentration is subsequently added to the prepared tissue. After an incubation time of about 10 minutes the tissue parts where digestion has started, are homogenized through passage through a Pasteur pipette.

Next, the tissue is brought into contact with an effective amount of the expansion medium of the invention, which has a Ca content of 0.02 mmol/l (in the form of CaCl₂), and a magnesium content of 0.4 mmol/l (as MgCl₂ or MgSO₄). Furthermore, the expansion medium contains the usual amounts of further components such as the ones cited above for an illustrative culture medium. As growth factors were comprised: inositol, EGF; FGF and LIF. Further commonly used compounds in culture media such as insulin, cortisone, penicillin and streptomycin and the like were present in usual concentrations. The culture medium is free of serum and serum extract, apart from human albumin, which is approved for the application in humans.

The expansion occurs under an atmosphere with reduced oxygen content of 0.1 to 5 Vol.-% (the norm value is 3%), and a CO₂ content of 5-10 Vol.-%, preferably 1-5 Vol.-% (norm value 5%), supplemented by 90-94% N₂ (norm value 92%).

The expanded tissue is homogenized by passage through an Eppendorf pipette and the cell number is determined using a hemocytometer. The cell suspension which is comprised virtually exclusively of individual cells and lose cell aggregates, is diluted with an expansion medium to a cell number of about 300.000 cells/ml. 8 ml of this cell suspension is pipetted into a 25 cbm bottle and the cells are cultivated in an atmosphere consisting of 1-5 Vol.-% oxygen, 5-10 Vol.-% CO₂ and 84-94 Vol.-% N₂ at 37° C.

The cells are fed one to two times per week with fresh expansion medium according to the invention having a calcium content of about 0.05 mmol/l expansion medium, for this the cells are transferred to plastic tubes and subsequently centrifuged. The supernatant is aspirated and 2 ml of fresh expansion medium is added, followed by homogenization. The homogenized solution is divided into several samples, passaged in fresh bottles and 8 ml of the expansion solution low on calcium is added.

The neural progenitor cells which were expanded in this way may be deep-frozen for storage in liquid nitrogen in the usual way. For this, the cell preparation occurs as usual (it is hereto referred to the disclosure of WO 00/78931 which is hereby incorporated by reference in its entirety). The cells are then also taken up in a suspension medium with a calcium content of about 0.05 mmol/l culture medium. For the partial differentiation of the progenitor cells, the frozen preparation may be thawed in a water bath and the cryotube can be disinfected with ethanol 70%. The cell suspension is slowly mixed with an expansion medium containing about 0.05 mmol Ca²⁺/l expansion medium under shaking, centrifuged again and taken up into expansion medium. Then, the sample is incubated for a week in a culture bottle under an atmosphere of 5 Vol.-% CO_{2/95}% air (preferably 3% oxygen and 92% nitrogen) at 37° C. The further expansion of cells occurs as described supra.

A partial differentiation of the progenitor cells can take place with freshly expanded or with thawed samples. Concerning the handling of the thawed samples and the use of the medium, it is referred to the “adding medium 1” of the disclosure of WO 00/78931, which is included hereby by reference in its entirety. The medium, which corresponds essentially to the “add in medium” disclosed in WO 00/78931 also has a calcium ion concentration of <0.1 mmol/l culture medium.

The samples are further processed according to variation a) or b)

Variation a)

The samples are then incubated for 7 to 21 days under an atmosphere having an oxygen concentration of 2 Vol.-%. The resulting cells are then selectioned through subcloning in an atmosphere having an oxygen concentration of 5 Vol.-%, whereby the above described expansion and partial differentiation steps are repeated.

The resulting cell suspensions can be taken up into a phosphate buffered salt solution for transplantation.

We obtained preparations of dopaminergic neurons, which are essentially free of glial cells. The cells can be transplanted in turn.

As a variation of the above described example, a culture medium having a Ca concentration of about 0,1 mmol/l culture medium and having a concentration of N- and E-Cadherin inhibition through N— and E-Cadherin Antisense and antibodies of 1 μg/ml culture medium, was used for the expansion of the cell culture. The method lead to the essentially same result, in particular a DNA fragmentation of less than 1% due to beginning apoptosis was observed.

Variation b)

The samples are then incubated for the partial differentiation using priming for 6 hours in the presence of IL-1 alpha (10 ng/ml) in an atmosphere having an oxygen content of 3 Vol.-%, and the medium is removed for the partial differentiation.

Then, the resulting cells are selectioned through subcloning in an atmosphere having an oxygen concentration of 3 Vol.-%, and then the supra described steps of expansion and partial differentiation can be repeated for an efficiency increase of the method.

The resulting cell suspension may be taken up into a phosphate buffered salt solution for transplantation. After the final differentiation through a 10 day incubation in the presence of differentiation medium, as described above, we obtained preparations of dopaminergic neurons which are essentially free of glial cells.

In this way, after a one-time application of the steps of partial differentiation through priming in the presence of lowered oxygen content, selectioning and subsequent expansion in a final differentiation step, about 15% of the obtained monoclonal cell lines differentiate each over 90% into dopaminergic neurons; after a repetition of the method steps of partial differentiation, selection and expansion, at the subsequent final differentiation about 21% of the monoclonal cell lines differentiate to 95% into dopaminergic neurons. The DNA fragmentation due to beginning apoptosis was less than 1%. The telomerase activity of the cells, determined as described supra, corresponded to FIG. 1 with use of corresponding Ca²⁺ concentration.

The specificity of the method can be further increased by applying a cell sorting step after then expansion (especially using IL-1 alpha receptor antibodies) and after the partial differentiation (especially using NCAM antibodies, as described supra).

1. A method for the cultivation of cell cultures comprising a plurality of neuronal progenitor cells, wherein said method comprises an expansion of the progenitor cells in a culture medium, wherein during the expansion the neuronal progenitor cells of the cell culture are at a substantial proportion present in the form of single cells and/or agglomerates with less than 100 cells per agglomerate, which can be dissociated into single cells by weak mechanical influences upon the culture medium without damage to the majority of the neuronal progenitor cells, wherein the expansion is carried out in a culture medium having a Ca²⁺ concentration of 0.001 to 0.5 mmol/l culture medium.

2. The method of claim 1, wherein the Mg²⁺ concentration of the culture medium is ≦2 mmol/l culture medium, preferably ≦0.6 mmol/l culture medium.

3. The method of claim 1 or 2, wherein the method step occurs in the presence of inhibitors which are specific for cellular receptors forming intercellular interactions.

4. The method of claim 3, wherein the method step occurs in the presence of inhibitors which are specific for at least one substance selected from the group of cadherins, selectins, integrins and immunoglobulins.

5. The method of claim 4, wherein the method step occurs in presence of one or more of the compounds eNCAM, L1, N-Cadherin.

6. The method of any of claims 1 to 5, wherein the method step occurs in presence of active telomerase.

7. The method of any of claims 1 to 6, wherein the method step occurs in a serum free and/or serumextract-free expansion medium.

8. The method of any one of claims 1 to 7, characterized in that in an initial step neuronal progenitor cells which are present in the form of spheroids, are transferred to a culture medium having a Ca²⁺ concentration of 0.001 to 0.5 mM, and that the progenitor cells remain for a sufficiently long time in said culture medium, preferably before a further method step is carried out, until the progenitor cells are present in the form of single cells or agglomerates consisting of less than 100 cells per agglomerate, which can be dissociated into single cells by weak mechanical influences upon the culture medium without damage to the majority of the progenitor cells.

9. The method of any of claims 1 to 8, characterized in that the method comprises one or more further method steps, especially selected from the group of partial differentiation, subcloning and priming, wherein progenitor cells are subjected to said one or more further method steps.

10. The method of claim 9, characterized in that the further method step is carried out with a cell culture, which at a substantial proportion contains single cells and/or agglomerates with weak intercellular interactions.

11. A method for the preparation of an expandable cell culture of progenitor cells, comprising the following method steps:
obtaining brain parts of a mammal
selection of progenitor cells
expansion of progenitor cells
partial differentiation of progenitor cells
if needed one or several repetitions of one or more of the steps expansion, selection and/or partial differentiation,
characterized in that during the expansion the neuronal progenitor cells of the cell culture are at a substantial proportion present in the form of single cells and/or agglomerates with less than 100 cells per agglomerate, which can be dissociated into single cells by weak mechanical influences upon the culture medium without damage to the majority of the neuronal progenitor cells, wherein the expansion is carried out in a culture medium having a Ca²⁺ concentration of 0.001 to 0.5 mmol/l culture medium.

12. A cell culture comprising a plurality of cells that are progenitor cells, wherein the progenitor cells are at a substantial proportion present in the form of single cells and/or agglomerates with less than 100 cells per agglomerate, which can be dissociated into single cells by weak mechanical influences upon the culture medium without damage to the majority of the neuronal progenitor cells; wherein the telomerase inhibition is less than 90% in relation to the fully active telomerase in the culture medium.

13. The cell culture of claim 12, wherein the neuronal progenitor cells have a telomerase activity of more than 20% of the telomerase activity of the control sample of the tumor cell line A549.

14. A cell culture comprising a plurality of cells that are neuronal cells, wherein the neuronal cells are at a substantial proportion present in the form of single cells and/or agglomerates with less than 100 cells per agglomerate, which can be dissociated into single cells by weak mechanical influences upon the culture medium without damage to the majority of the neuronal cells; wherein the telomerase inhibition is less than 90% in relation to the fully active telomerase in the culture medium.

15. The cell culture of any of claims 12 to 14, wherein the progenitor cells and/or neuronal cells are present in a concentration of at least 10,000 cells/ml culture medium, in the form of single cells and/or agglomerates with less than 100 cells per agglomerate.

16. A cell culture, especially according to any of claims 12 to 15, isolated after execution of one or more method steps of claims 1 to 11, and optionally of further method steps.

17. The cell culture of any of the claims 12 to 16 in a form suitable for administration to an animal including humans.

18. The use of a culture medium having a Ca²⁺ concentration of 0.001 to 0.5 mol/l culture medium for the preparation of a cell culture according to any of the claims 12 to 17.
